(19) United States
(12) Patent Application Publication
(10) Pub. No.: US 2024/0096493 A1
(43) Pub. Date: Mar. 21, 2024

(54) COMPUTATIONAL SYSTEM AND METHOD FOR DIAGNOSIS, PROGNOSIS, AND THERAPEUTICS OF CANCER PATIENTS USING SPATIAL-TEMPORAL TISSUE ARCHITECTURAL PROPERTIES

(71) Applicants: THE BOARD OF REGENTS OF THE UNIVERSITY OF TEXAS SYSTEM, Austin, TX (US); BOARD OF REGENTS OF THE UNIVERSITY OF NEBRASKA, Lincoln, NE (US)

(72) Inventors: Matteo Ligorio, Dallas, TX (US); Rebecca Napier, Boynton Beach, FL (US); William Gasper, Omaha, NE (US); Dario Ghersi, Omaha, NE (US)

(73) Assignees: THE BOARD OF REGENTS OF THE UNIVERSITY OF TEXAS SYSTEM, Austin, TX (US); BOARD OF REGENTS OF THE UNIVERSITY OF NEBRASKA, Lincoln, NE (US)

(21) Appl. No.: 18/469,022

(22) Filed: Sep. 18, 2023

Related U.S. Application Data
(60) Provisional application No. 63/376,558, filed on Sep. 21, 2022.

(51) Int. Cl. G16H 50/20; G06T 7/00; G06T 7/50; G16H 50/50; G16H 70/60

(52) U.S. Cl. CPC G16H 50/20; G06T 7/0012; G06T 7/50; G16H 50/50; G16H 70/60; G06T 2207/20021; G06T 2207/20036; G06T 2207/20081; G06T 2207/30096; G06T 2207/30242

(57) ABSTRACT

Aspects of the present disclosure relate to systems and methods for analyzing a tumor, and more specifically, analyzing a tumor to identify a biological aggressiveness of the tumor. One example method for tumor analysis includes: receiving one or more input features associated with a geometric shape of a tumor, the one or more input features including at least a value indicating a circularity of the tumor; determining, via a machine learning component, a characteristic of the tumor based on the one or more input features, the characteristic being indicative of patient survivability; and outputting an indication of the characteristic.

### CROSS-REFERENCE TO RELATED APPLICATION

This application claims priority to U.S. Provisional Patent Application Ser. No. 63/376,558 filed Sep. 21, 2022, and titled “COMPUTATIONAL SYSTEM AND METHOD FOR DIAGNOSIS, PROGNOSIS, AND THERAPEUTICS OF CANCER PATIENTS USING SPATIAL-TEMPORAL TISSUE ARCHITECTURAL PROPERTIES,” the entirety of which is incorporated herein by reference.

### ACKNOWLEDGEMENT OF GOVERNMENT SUPPORT

This invention was made with government support under Grant No. CA242070 awarded by the National Institutes of Health (NIH). The government has certain rights in the invention.

### BACKGROUND

### 1. Technical Field

Aspects of the present disclosure relate to systems and methods for analyzing a tumor, and more specifically, analyzing a tumor to identify a biological aggressiveness of the tumor.

### 2. Discussion of Related Art

Tumors are groups of abnormal cells that form lumps or growths. They can start in any one of the trillions of cells in our bodies. Tumors grow and behave differently depending on whether they are cancerous (malignant), non-cancerous (benign) or precancerous. Benign tumors may grow large but do not spread into or invade nearby tissues or other parts of the body. Malignant tumors can spread into, or invade, nearby tissues.

### SUMMARY

Certain aspects are directed towards a system for tumor analysis. The system can include a memory and one or more processors coupled to the memory. The memory may include instructions which, when executed by the one or more processors, cause the one or more processors to: receive one or more input features associated with a geometric shape of a tumor, the one or more input features including at least a value indicating a circularity of the tumor; determine, via a machine learning component, a characteristic of the tumor based on the one or more input features, the characteristic being indicative of patient survivability; and output an indication of the characteristic.

In some examples, the instructions further cause the one or more processors to compute the value indicating the circularity of the tumor based on an area of the tumor divided by an area of a circle having a same perimeter length as the tumor. Also, the one or more input features can further include a value indicating a discrepancy between a shape of the tumor and a shape of a lumen associated with the tumor. The one or more input features can further include a value indicating an eccentricity of the tumor. Additionally, the tumor can be one of a plurality of neighboring tumors, and/or the one or more input features further include a value indicating a coherence between orientations of the plurality of neighboring tumors. Moreover, the instructions can further cause the one or more processors to calculate the value indicating the coherence based on an angle between principal axes of at least two of the plurality of neighboring tumors. Furthermore, the tumor can be one of a plurality of tumors, and/or the one or more input features can include a quantity of the plurality of tumors that have a number of whitespace features great than a threshold. Also, the one or more input features can include a number of instances of cytoplasmic vacuolization, signet ring cells, and shearing detected for the tumor. The one or more input features can also include a measure of an evolution of an architecture of the tumor. Additionally, the machine learning component can be trained using a labeled dataset associated with geometric shapes of tumors. Also, to output the indication of the characteristic, the one or more processors can be configured to output an indication of a biological aggressiveness of the tumor.

Certain aspects are directed towards a method for tumor analysis. The method generally includes: receiving one or more input features associated with a geometric shape of a tumor, the one or more input features including at least a value indicating a circularity of the tumor; determining, via a machine learning component, a characteristic of the tumor based on the one or more input features, the characteristic being indicative of patient survivability; and outputting an indication of the characteristic.

In some examples, the method further includes computing the value indicating the circularity of the tumor based on an area of the tumor divided by an area of a circle having a same perimeter length as the tumor. Moreover, the one or more input features can further include a value indicating a discrepancy between a shape of the tumor and a shape of a lumen associated with the tumor. Furthermore, the one or more input features can further include a value indicating an eccentricity of the tumor. The tumor can be one of a plurality of neighboring tumors, and/or the one or more input features can further include a value indicating a coherence between orientations of the plurality of neighboring tumors. The method can also include calculating the value indicating the coherence based on an angle between principal axes of at least two of the plurality of neighboring tumors. Additionally, the tumor can also be one of a plurality of tumors, and/or the one or more input features can include a quantity of the plurality of tumors that have a number of whitespace features great than a threshold. Also, the one or more input features can include a number of instances of cytoplasmic vacuolization, signet ring cells, and shearing detected for the tumor.

Certain aspects are directed towards a non-transitory computer-readable medium having instructions stored thereon, that when executed by one or more processors, cause the one or more processors to: receive one or more input features associated with a geometric shape of a tumor, the one or more input features including at least a value indicating a circularity of the tumor; determine, via a machine learning component, a characteristic of the tumor based on the one or more input features, the characteristic being indicative of patient survivability; and output an indication of the characteristic.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 illustrates an example computing device, in accordance with certain aspects of the present disclosure.

FIG. 2 illustrates a group of cancer cells identified for analysis, in accordance with certain aspects of the present disclosure.

FIGS. 3A and 3B illustrate the computation of a value indicating a circularity of a tumor gland, in accordance with certain aspects of the present disclosure.

FIG. 4 shows pancreatic ducts where the shape of the duct closely mirrors the shape of the lumen, in accordance with certain aspects of the present disclosure.

FIG. 5A shows a tumor gland having a low discrepancy between the lumen and whitespace shape and the tumor gland shape.

FIG. 5B shows a tumor gland having a high discrepancy between the lumen and whitespace shape and the tumor gland shape.

FIG. 5C is a plot showing survival probabilities over time for high and low risk groups, as assessed based on predicted tumor and lumen shape discrepancies, in accordance with certain aspects of the present disclosure.

FIG. 6A illustrates a tumor gland with extensive cytoplasmic vacuolization.

FIG. 6B illustrates results of an unsupervised technique for detecting intra-gland whitespace features, in accordance with certain aspects of the present disclosure.

FIG. 7 is a plot showing survival probabilities over time for high and low risk groups of patients, as assessed based on the number of tumor glands with pervasive cytoplasmic vacuolization, signet ring cells, and shearing, in accordance with certain aspects of the present disclosure.

FIG. 8 illustrates two elongated tumor glands, and representations of their principal axes of inertia, in accordance with certain aspects of the present disclosure.

FIG. 9 is a plot showing survival probabilities over time for high and low risk groups, as assessed based on the proportion of highly eccentric tumor glands, in accordance with certain aspects of the present disclosure.

FIG. 10 is a plot showing survival probabilities over time for high and low risk groups, as assessed based on the proportion of neighboring glands with orientation discrepancies and agreements, in accordance with certain aspects of the present disclosure.

FIG. 11 is Kernel density plots showing differences in angle distributions for neighboring tumor gland pairs and dispersed gland pairs at varying distances with sufficient eccentricity to have orientation.

FIG. 12 shows Spearman correlations between gland pair angles and distances as minimum eccentricity values are increased.

FIG. 13 is a flow diagram illustrating example operations for tumor analysis, in accordance with certain aspects of the present disclosure.

It will be apparent to one skilled in the art after review of the entirety disclosed that the steps illustrated in the figures listed above may be performed in other than the recited order, and that one or more steps illustrated in these figures may be optional.

### DETAILED DESCRIPTION

Tissue architecture has been shown as playing an important role in determining tumor aggressiveness with a large effect on patient prognosis. Certain aspects of the present disclosure provide a system for extracting tangible (e.g., easily interpretable) features from images of solid malignancies stained with hematoxylin and eosin (H&E) to determine the presence of tumors cells (e.g., improving diagnosis) and to estimate tumor aggressiveness (e.g., tumor grade or vascular invasion). The aspects described herein provide a prognostication tool and techniques for stratifying patients to identify the ones who respond to chemotherapy, targeted therapy, and immunotherapy.

The tumor analysis system provided herein uses large-scale multiple geometric and non-geometric measures computed directly from annotated H&E slides and can be directly integrated with different spatial omics technologies. Some examples of geometric features are described below together with the associated preliminary data, which can be applied to both primary tumors and distant metastases.

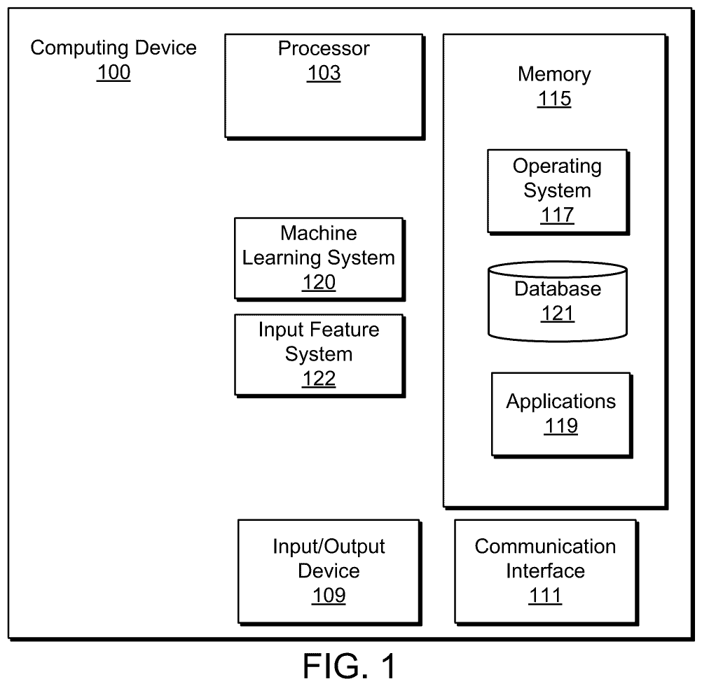

FIG. 1 illustrates an example computing device 100, in accordance with certain aspects of the present disclosure. The computing device 100 can include a processor 103 for controlling overall operation of the computing device 100 and its associated components, including input/output device 109, communication interface 111, and/or memory 115. A data bus can interconnect processor(s) 103, memory 115, I/O device 109, and/or communication interface 111.

Input/output (I/O) device 109 can include a microphone, keypad, touch screen, and/or stylus through which a user of the computing device 100 can provide input and can also include one or more of a speaker for providing audio output and a video display device for providing textual, audiovisual, and/or graphical output. Software can be stored within memory 115 to provide instructions to processor 103 allowing computing device 100 to perform various actions for tumor analysis. Memory 115 can store software used by the computing device 100, such as an operating system 117, application programs 119, and/or an associated internal database 121.

Communication interface 111 can include one or more transceivers, digital signal processors, and/or additional circuitry and software for communicating via any network, wired or wireless, using any protocol as described herein. Processor 103 can include a single central processing unit (CPU), which can be a single-core or multi-core processor (e.g., dual-core, quad-core, etc.), or can include multiple CPUs. Processor(s) 103 and associated components can allow the computing device 100 to execute a series of computer-readable instructions to perform some or all of the processes described herein.

As shown, the computing device 100 may include an input feature computation system 122, which may calculate one or more input features to be processed by a machine learning system. For instance, the computing device 100 may include a machine learning system 120 that receives the input features and outputs a characteristic of a tumor based on the input features. The characteristic of the tumor may indicate a biological aggressiveness of the tumor.

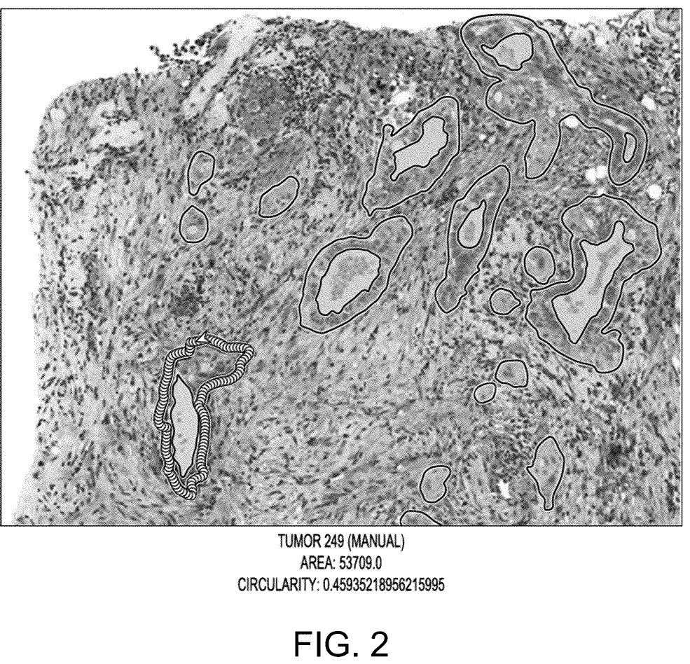

FIG. 2 illustrates a group of cancer cells identified for analysis, in accordance with certain aspects of the present disclosure. The tumor analysis system may identify a single or group of cancer cells in defined anatomical structures (e.g., tumor glands) and their internal lumen architecture using machine learning techniques in H&E stained tissue slide scans. As data, these tumor and lumen features exist as polygons represented by lists of consecutive points, as shown in FIG. 2. Using the identified tumor glands, one or more input features may be calculated. One example of an input feature may include a circularity of tumor glands.

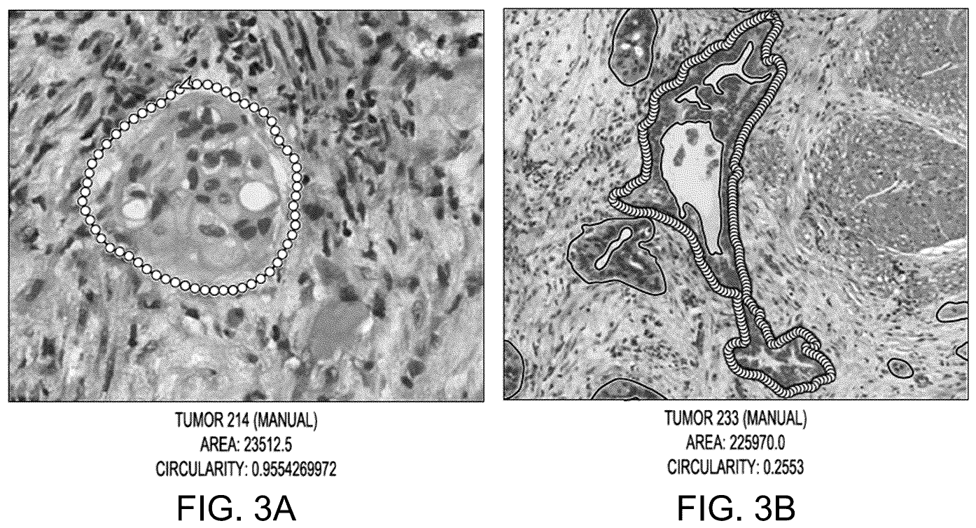

FIGS. 3A and 3B illustrate computation of a value indicating a circularity of a tumor gland, in accordance with certain aspects of the present disclosure. The analysis system may compute the area of the tumor gland divided by the area of a perfect circle with the same perimeter length as the tumor gland. The value indicating the circularity of a tumor gland may range from 1 for perfectly circular tumor glands to near zero for glands that are highly non-circular. FIG. 3A shows a highly circular tumor and FIG. 3B shows a non-circular tumor. The high circular tumor gland may have a circularity value of 0.955 and the non-circular tumor gland may have a circularity value of 0.255.

Certain aspects provide identification of tumor features (diagnosis) by automatically identifying tumor structures and linked features in histopathologic H&E imaging using machine learning. A discrepancy between lumen and whitespace shape and the tumor gland shape may be considered for tumor analysis.

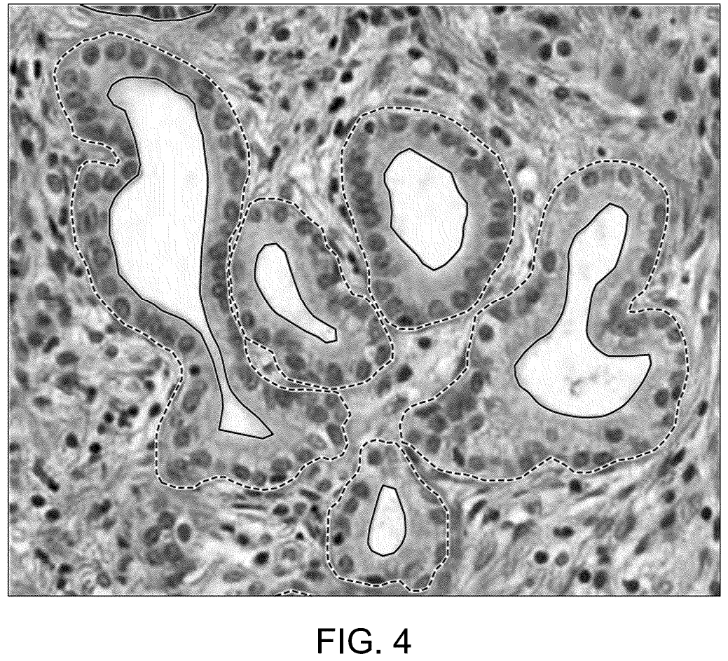

FIG. 4 shows pancreatic ducts where the shape of the duct closely mirrors the shape of the lumen, in accordance with certain aspects of the present disclosure. A relationship exists between shape discrepancies (with regard to each tumor gland and its whitespace features) and overall patient survival. For example, in healthy pancreatic ducts, the shape of the duct closely mirrors the shape of the lumen, as shown.

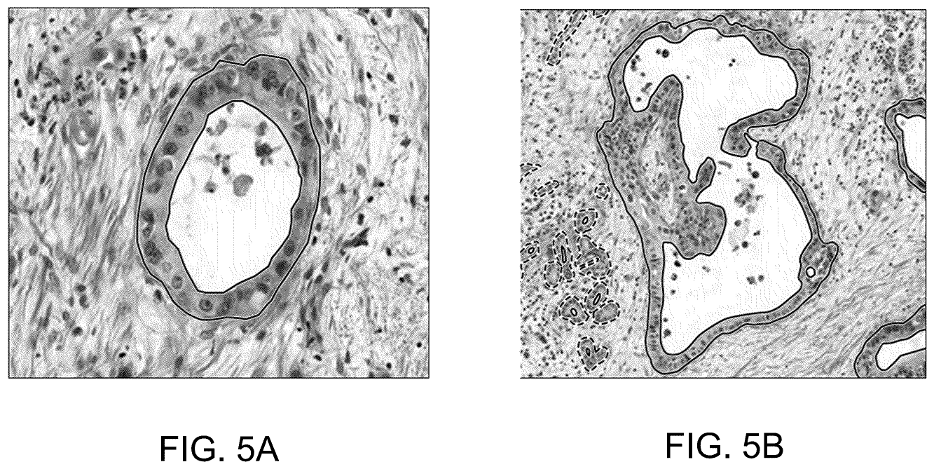

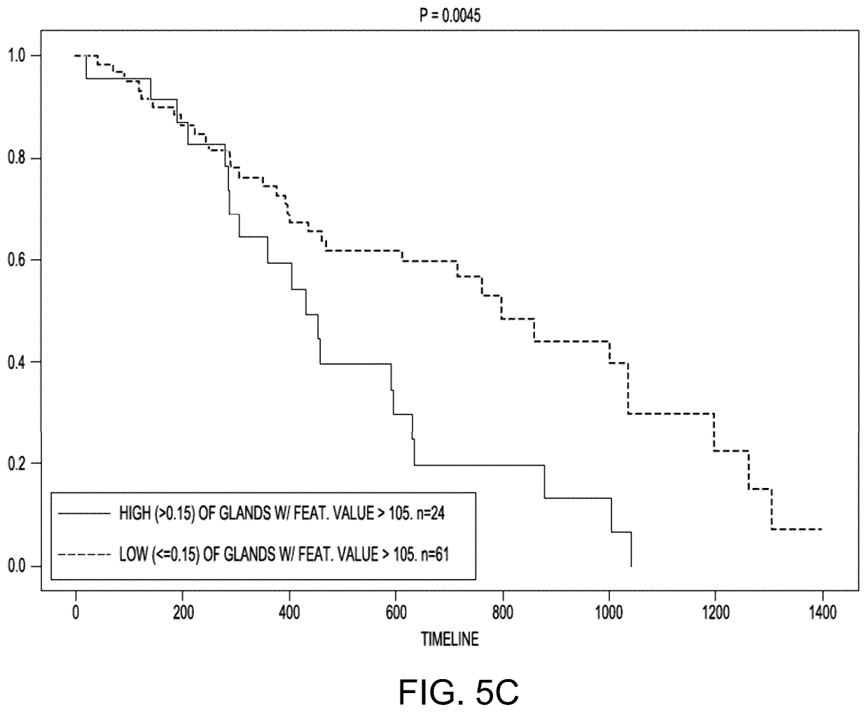

FIG. 5A shows a tumor gland having a low discrepancy between the lumen and whitespace shape and the tumor gland shape. FIG. 5B shows a tumor gland having a high discrepancy between the lumen and whitespace shape and the tumor gland shape. In some aspects, the input feature computation system computes the numerical representations of each tumor gland shape and the shape of its respective lumen(s) and whitespace features, which may be caused by damage (shearing and cytoplasmic vacuolization). Then, the system may compute a distance metric to describe the discrepancy between the shape of a tumor gland and its lumen(s).

FIG. 5C is a plot showing survival probabilities over time for high and low risk groups, as assessed based on predicted tumor and lumen shape discrepancies, in accordance with certain aspects of the present disclosure. The plot shows a relationship between patient survival and glands having high discrepancies values of tumor gland shape and the shape of its respective lumen(s) and whitespace features. As shown, a statistically significant relationship exists between the proportion of glands with high discrepancies values and patient survival (e.g., a p-value of 0.00045). A p-value is a statistical measurement used to validate a hypothesis against observed data. A p-value measures the probability of obtaining the observed results, assuming that the null hypothesis is true. The lower the p-value, the greater the statistical significance of the observed difference.

Another input feature that may be identified and used for tumor analysis may include cytoplasmic vacuolization, signet ring, and shearing damage. The input feature calculation system may detect instances of cytoplasmic vacuolization, signet ring cells, and shearing due to tissue friability (e.g., indicators of cellular distress) within tumor glands and record the number of instances for each tumor gland.

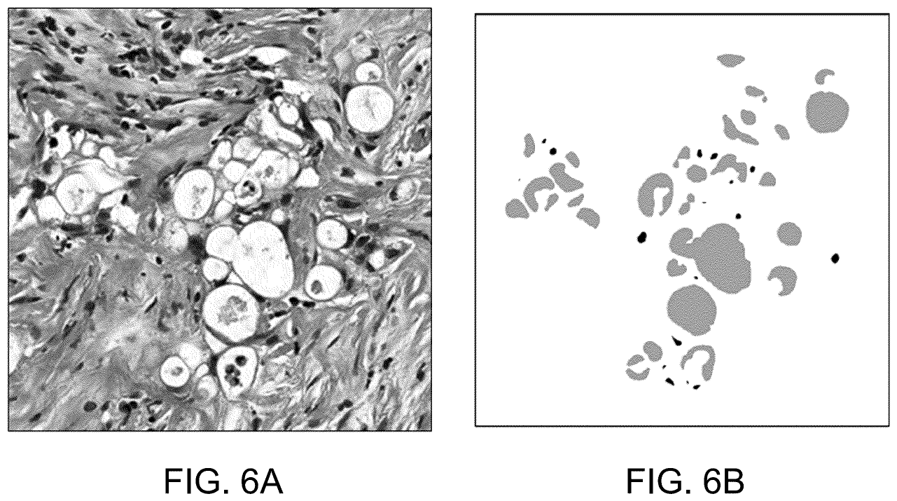

FIG. 6A illustrates a tumor gland with extensive cytoplasmic vacuolization. Vacuoles are areas of the cytoplasm which do not stain with Wright's stain and appear as holes in the cytoplasm. FIG. 6B illustrates results of an unsupervised technique for detecting intra-gland whitespace features, in accordance with certain aspects of the present disclosure. The technique involves isolating tumor gland pixels, binarizing the tumor gland, and then blurring the image, using a step activation function, and finding contours within the resulting representation. After using a minimum area threshold, meaningful whitespace features are identified, as shown in FIG. 6B. Meaningful whitespace features are shown in gray. Some features are excluded due to their size (e.g., not meeting the minimum area threshold), which are shown in white in FIG. 6B. The number of tumor glands with high numbers of whitespace features, which when greater than a threshold (e.g., 9) tend to be vacuoles, signet ring cells, or shearing (indicators of cellular distress), correlates with patient survival.

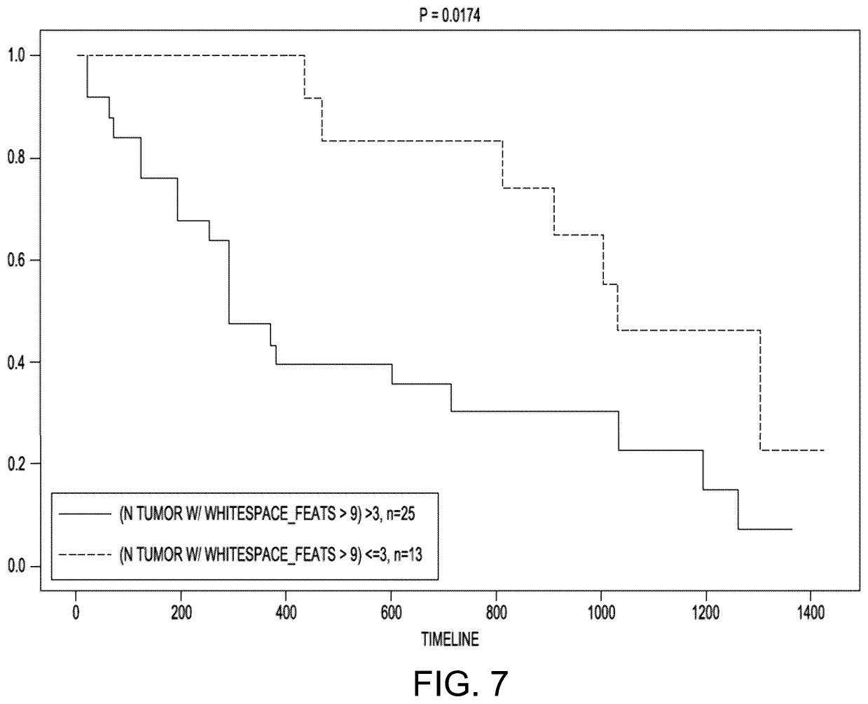

FIG. 7 is a plot showing survival probabilities over time for high and low risk groups of patients, as assessed based on the number of tumor glands with pervasive cytoplasmic vacuolization, signet ring cells, and shearing, in accordance with certain aspects of the present disclosure. As shown, a statistically significant relationship exists between patient survival and the number of tumor glands with pervasive cytoplasmic vacuolization, signet ring cells, and shearing (e.g., a p-value of 0.0174).

In some aspects, the input feature calculation system may identify tumor gland eccentricity. By computing the principal axes of tumor glands, the system identifies tumor glands that are highly elongated about their centroid. The elongation may be determined by comparing the ratio of the variances in the principal axes. The proportion of highly elongated glands for each patient can then be assessed and used as an input feature to the machine learning system. The system examines orientation and eccentricity of tumor glands, a relative measure of elongation about the center of mass of a feature. By computing the principal axes of inertia, the system determines the orientation of elongated tumor glands.

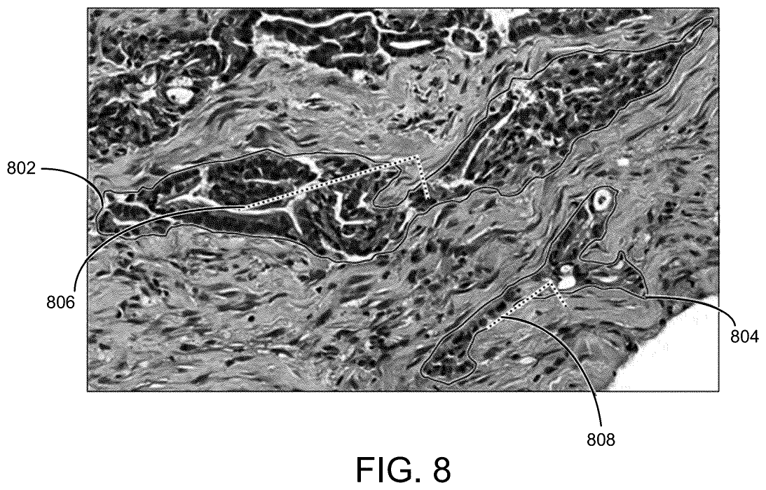

FIG. 8 illustrates two elongated tumor glands 802, 804, and representations of their principal axes of inertia, shown by lines 806, 808, respectively, in accordance with certain aspects of the present disclosure. The glands are elongated and also have high eccentricity. Eccentricity values describe relative elongation about the centroid of the structure. A relationship exists between having a small proportion of high eccentricity (e.g., extremely elongated) glands and patient survival.

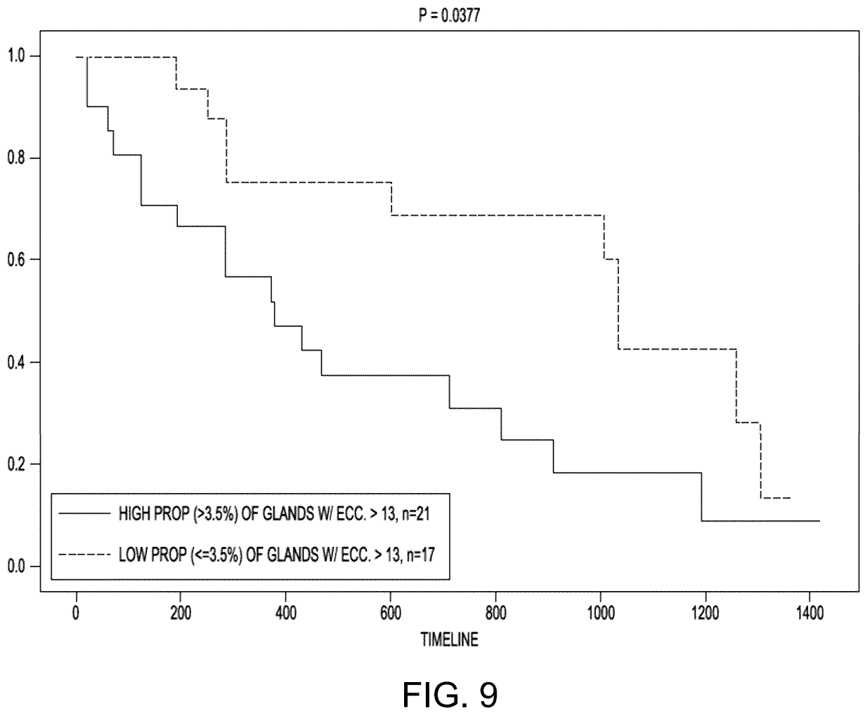

FIG. 9 is a plot showing survival probabilities over time for high and low risk groups, as assessed based on the proportion of highly eccentric (elongated about the centroid) tumor glands, in accordance with certain aspects of the present disclosure. As shown, a statistically significant relationship exists between patient survival and the proportion of highly eccentric (elongated about the centroid) tumor glands (e.g., a p-value of 0.0377).

An example of an input feature that may be used may include tumor vector fields calculated as a function of a two-dimensional geometry of tumor structures. The system may compute the principal axes of tumor glands that are present in an H&E slide. The system then calculates an index that expresses the coherence of tumor gland orientation. This is achieved by computing the angle between each major principal axis for neighboring tumor glands. The system then assesses neighboring gland orientation with a representative metric for each patient. These tumor vector fields can also be enriched by genetic, transcriptomic and proteomics elements to better define their biological implications. For example, referring back to FIG. 8, the principal axes of tumor glands 802, 804 are shown by lines 806, 808, respectively. The difference in the angles of the principal axes of the tumor glands may be used to generate the index expressing the coherence of tumor gland orientation.

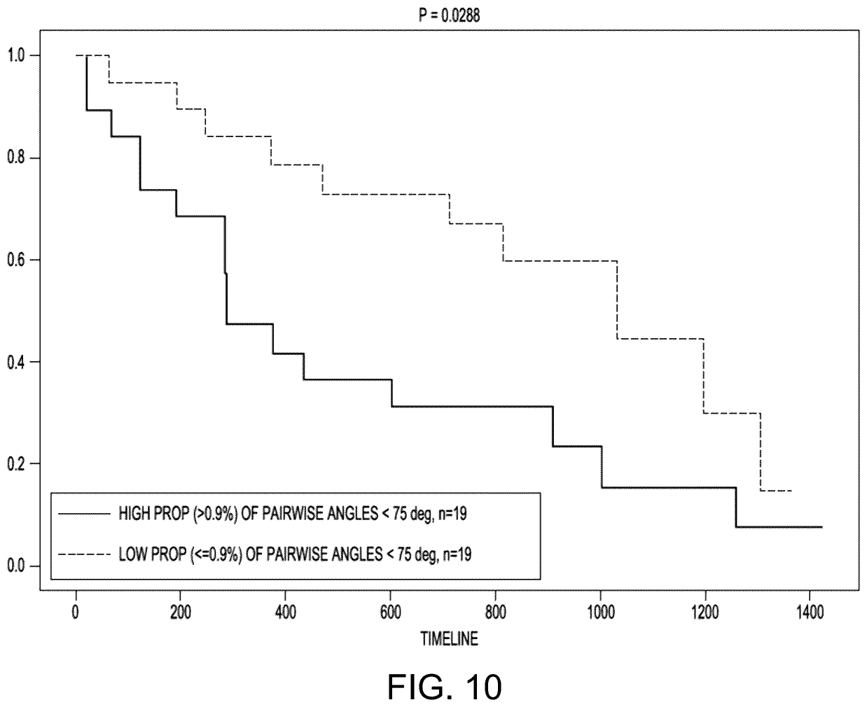

FIG. 10 is a plot showing survival probabilities over time for high and low risk groups, as assessed based on the proportion of neighboring glands with orientation discrepancies and agreements, in accordance with certain aspects of the present disclosure. As described, the system calculates angles between principal axes (tumor vector fields) in order to determine coherence in orientation for neighboring, elongated glands. Patients with a deviation in coherence for a proportion of neighboring glands live longer than those patients with more coherence for neighboring glands, as shown. This result is more significant as a continuous variable (p=0.01) in Cox regression, indicating that patient survival increases as the proportion of neighboring gland angle less than 75 degrees decreases.

In some aspects, an input feature may be based on tumor vector meta-structures. These higher-order structures are calculated from the local tumor vector fields and can be interpreted as the spatio-temporal evolution of tumor tissue architectures.

The methodology described herein works effectively in an automated manner that is not sensitive to certain characteristic segmentation prediction errors. The segmentation model (machine learning to identify tumor and other structures automatically) may be trained on a relatively small amount of data.

The prior variables may be combined into a multivariate Cox regression and produce the following results, including hazards ratio (H.R.), lower 95% H.R. confidence limit, upper 95 H.R. confidence limit, and p-value.

| | | H.R. | H.R. | |
|---|---|---|---|---|
| Variable | H.R. | lower 95% | upper 95% | p |
| Whitespace features | 1.06 | 1.01 | 1.12 | 0.028 |
| (vacuoles, signet rings, etc.) | | | | |
| Eccentricity, binned | 1.85 | 0.82 | 4.15 | 0.149 |
| Angle feature | 1.64e5 | 21.3 | 1.27e9 | 0.008 |

In this case, the whitespace feature and angle features are left continuous, and the eccentricity feature is discretized to divide patients into two groups. Patients may be binned into two groups for whitespace features and angle features, and this produces the following result:

| | | H.R. | H.R. | |
|---|---|---|---|---|
| Variable | H.R. | lower 95% | Upper 95% | p |
| Whitespace features, binned | 3.34 | 1.33 | 8.36 | 0.010 |
| Eccentricity, binned | 2.31 | 1.01 | 5.27 | 0.0477 |
| Angle feature, binned | 2.25 | 1.01 | 5.02 | 0.0482 |

Tumor vector field meta-structures composed of groups of neighboring tumor glands with similar spatial orientations may be identified and quantified.

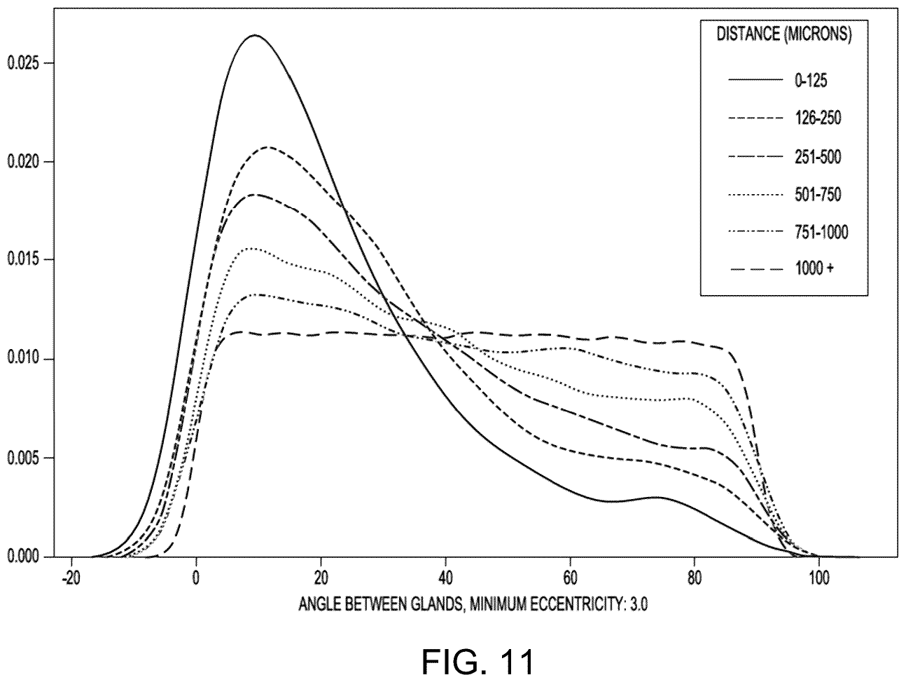

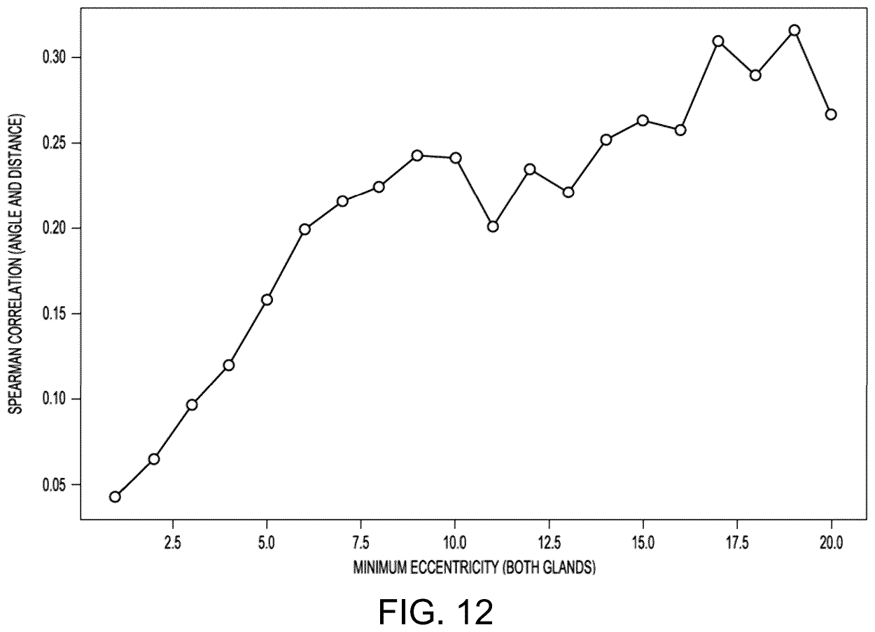

FIG. 11 is Kernel density plots showing differences in angle distributions for neighboring tumor gland pairs and dispersed gland pairs at varying distances with sufficient eccentricity to have orientation. FIG. 11 demonstrates that neighboring glands with sufficient eccentricity to have a directional orientation are more aligned, with respect to orientation, than dispersed glands. FIG. 12 shows Spearman correlations between gland pair angles and distances as minimum eccentricity values are increased. FIG. 12 demonstrates that as glands become more elongated about their centroid, the relationship between distance and angle becomes stronger; this relationship between minimum eccentricity and distance/angle correlation is highly correlated (Pearson's r=0.905, p=4.15e-8). Thus, the data supports the existence of higher-order tumor vector field structures.

Deep learning-based methods using imaging for diagnosis, metastasis detection, prognosis determination, and a number of other outcomes may be used. The techniques described herein are driven by interpretable measures and data that can be used to generate biological hypotheses with the intent to produce interpretable discoveries and to predict response to therapies and patient prognosis. In contrast, approaches purely based on deep learning tend to produce black boxes that, while performing well in some cases, are not interpretable. Any machine learning methods relating features (images) to a specific outcome are also prone to overfitting, may capture characteristics specific to the training data, and may not perform effectively on data. The analytical aspect of the tumor analysis system described herein, in many respects, is agnostic to certain data novelty issues that may confound machine learning methods, as the system analyzes derived features that are universal and are unlikely to be confounded by changes in imaging characteristics like staining or alternate modalities.

The present system performs tumor analysis using data about tumor morphology from large whole slide images in ways that may not be possible with current deep learning methods. For example, the techniques described herein use whole slide images with approximately 120 million pixels (e.g., a ˜11000×˜11000 pixel image) and can readily accept larger images without any issue. Purely deep learning models are limited to comparatively smaller images, and even with state-of-art hardware, models may be limited to accepting 1-million pixel images. Down-sampling whole slide images is not desirable either, as down-sampling to an extreme degree reduces (or eliminates) cellular and tissue morphology features required to determine spatial-temporal tissue architectural features and properties (e.g., status of the cells and their tumor gland structures).

The techniques described herein capture morphology using deep learning models from crops of images, and are able to generate an analyzable representation of the tumor morphology for an entire slide. The system described herein performs analysis which can also lead to hypothesis generation with biologically interpretable results, a valuable aspect for researchers, clinicians, and trainees. The techniques described herein may help trainees with different levels of expertise, from high-school students to pathology fellows, to test their knowledge and acquire new skills in tumor pathology and the biological aggressiveness of the tumor.

Pathology is a cornerstone of modern cancer diagnosis and prognosis. Computer vision has the potential to transform what pathology can accomplish. More specifically, there is a great need for automated approaches that can aid pathologists in providing accurate diagnoses and in correctly estimating tumor aggressiveness for patient management.

The features described herein have predictive power in discriminating patients with poor prognoses from long-term survivors, revealing how direct and objective tumor geometry measurements are directly implicated in tumor progression. To this end, the association between these features and clinical outcomes also points towards a deep biological connection between tumor geometry, gene expression, and underlying genetic events. These findings indicate that, in addition to an automated clinical platform, the aspects of the present disclosure provide unique research and educational toolkit for high-throughput analysis of H&E-stained pathology images.

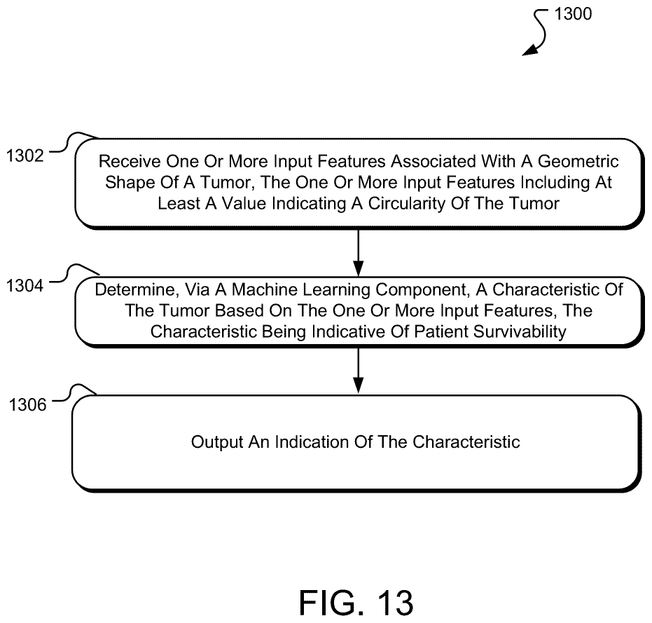

FIG. 13 is a flow diagram illustrating example operations 1300 for tumor analysis, in accordance with certain aspects of the present disclosure. The operations 1300 may be performed by, for example, a computing device such as the computing device 100 of FIG. 1.

The operations 1300 begin, at block 1302, with the computing device receiving one or more input features associated with a geometric shape of a tumor. In some aspects, the one or more input features may include at least a value indicating a circularity of the tumor. In some aspects, the computing device may compute (e.g., via input feature computation system 122 of FIG. 1) the value indicating the circularity of the tumor based on an area of the tumor divided by an area of a circle having a same perimeter length as the tumor.

In some aspects, the one or more input features may include a value indicating a discrepancy between a shape of the tumor and a shape of a lumen associated with the tumor. The value indicating the discrepancy may be calculated by the input feature computation system 122. In some cases, the one or more input features may include a value indicating an eccentricity of the tumor. The eccentricity may be determined or calculated by the input feature computation system 122.

In some cases, the tumor may be one of a plurality of neighboring tumors. In this case, the one or more input features may include a value indicating a coherence between orientations of the plurality of neighboring tumors (e.g., as calculated by the input feature computation system 122). In some cases, the computing device may calculate the value indicating the coherence based on an angle between principal axes of at least two of the plurality of neighboring tumors. In some aspects, the one or more input features may include a quantity of the plurality of tumors that have a number of whitespace features great than a threshold.

In some aspects, the one or more input features includes a number of instances of cytoplasmic vacuolization, signet ring cells, and shearing detected for the tumor. The one or more input features include a measure of an evolution of an architecture of the tumor

At block 1304, the computing device determines, via a machine learning component (e.g., machine learning system 120 of FIG. 1), a characteristic of the tumor based on the one or more input features, the characteristic being indicative of patient survivability. The machine learning component may be trained using a labeled dataset associated with geometric shapes of tumors.

At block 1306, the computing device outputs an indication of the characteristic. To output the indication of the characteristic, the computing device may output an indication of a biological aggressiveness of the tumor.

These and various other arrangements will be described more fully herein. As will be appreciated by one of skill in the art upon reading the following disclosure, various aspects described herein can be a method, a computer system, or a computer program product. Accordingly, those aspects can take the form of an entirely hardware implementation, an entirely software implementation, or at least one implementation combining software and hardware aspects. Furthermore, such aspects can take the form of a computer program product stored by one or more computer-readable storage media (e.g., non-transitory computer-readable medium) having computer-readable program code, or instructions, included in or on the storage media. Any suitable computer-readable storage media can be utilized, including hard disks, CD-ROMs, optical storage devices, magnetic storage devices, and/or any combination thereof. In addition, various signals representing data or events as described herein can be transferred between a source and a destination in the form of electromagnetic waves traveling through signal-conducting media such as metal wires, optical fibers, and/or wireless transmission media (e.g., air and/or space).

Implementations of the present disclosure include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software and/or firmware.

While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an implementation in the present disclosure can be references to the same implementation or any implementation; and, such references mean at least one of the implementations.

Reference to “one implementation” or “an implementation” means that a particular feature, structure, or characteristic described in connection with the implementation is included in at least one implementation of the disclosure. The appearances of the phrase “in one implementation” in various places in the specification are not necessarily all referring to the same implementation, nor are separate or alternative implementations mutually exclusive of other implementations. Moreover, various features are described which may be exhibited by some implementations and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various implementations given in this specification.

Without intent to limit the scope of the disclosure, examples of instruments, apparatus, methods and their related results according to the implementations of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

What is claimed is:

1. A system for tumor analysis comprising:
a memory; and
one or more processors coupled to the memory, the memory including instructions which, when executed by the one or more processors, cause the one or more processors to:
receive one or more input features associated with a geometric shape of a tumor, the one or more input features including at least a value indicating a circularity of the tumor;
determine, via a machine learning component, a characteristic of the tumor based on the one or more input features, the characteristic being indicative of patient survivability; and
output an indication of the characteristic.

2. The system of claim 1, wherein the instructions further cause the one or more processors to:
compute the value indicating the circularity of the tumor based on an area of the tumor divided by an area of a circle having a same perimeter length as the tumor.

3. The system of claim 1, wherein the one or more input features further include a value indicating a discrepancy between a shape of the tumor and a shape of a lumen associated with the tumor.

4. The system of claim 1, wherein the one or more input features further include a value indicating an eccentricity of the tumor.

5. The system of claim 1,
wherein,
the tumor is one of a plurality of neighboring tumors, and
the one or more input features further include a value indicating a coherence between orientations of the plurality of neighboring tumors.

6. The system of claim 5, wherein the instructions further cause the one or more processors to:
calculate the value indicating the coherence based on an angle between principal axes of at least two of the plurality of neighboring tumors.

7. The system of claim 1,
wherein,
the tumor is one of a plurality of tumors, and
the one or more input features include a quantity of the plurality of tumors that have a number of whitespace features great than a threshold.

8. The system of claim 1, wherein the one or more input features includes a number of instances of cytoplasmic vacuolization, signet ring cells, and shearing detected for the tumor.

9. The system of claim 1, wherein the one or more input features include a measure of an evolution of an architecture of the tumor.

10. The system of claim 1, wherein the machine learning component is trained using a labeled dataset associated with geometric shapes of tumors.

11. The system of claim 1, wherein, to output the indication of the characteristic, the one or more processors are configured to output an indication of a biological aggressiveness of the tumor.

12. A method for tumor analysis comprising:
receiving one or more input features associated with a geometric shape of a tumor, the one or more input features including at least a value indicating a circularity of the tumor;
determining, via a machine learning component, a characteristic of the tumor based on the one or more input features, the characteristic being indicative of patient survivability; and
outputting an indication of the characteristic.

13. The method of claim 12, further comprising:
computing the value indicating the circularity of the tumor based on an area of the tumor divided by an area of a circle having a same perimeter length as the tumor.

14. The method of claim 12, wherein the one or more input features further include a value indicating a discrepancy between a shape of the tumor and a shape of a lumen associated with the tumor.

15. The method of claim 12, wherein the one or more input features further include a value indicating an eccentricity of the tumor.

16. The method of claim 12,
wherein,
the tumor is one of a plurality of neighboring tumors, and
the one or more input features further include a value indicating a coherence between orientations of the plurality of neighboring tumors.

17. The method of claim 16, further comprising:
calculating the value indicating the coherence based on an angle between principal axes of at least two of the plurality of neighboring tumors.

18. The method of claim 12,
wherein,
the tumor is one of a plurality of tumors, and
the one or more input features include a quantity of the plurality of tumors that have a number of whitespace features great than a threshold.

19. The method of claim 12, wherein the one or more input features includes a number of instances of cytoplasmic vacuolization, signet ring cells, and shearing detected for the tumor.

20. A non-transitory computer-readable medium having instructions stored thereon, that when executed by one or more processors, cause the one or more processors to:
receive one or more input features associated with a geometric shape of a tumor, the one or more input features including at least a value indicating a circularity of the tumor;
determine, via a machine learning component, a characteristic of the tumor based on the one or more input features, the characteristic being indicative of patient survivability; and
output an indication of the characteristic.
